(19) United States
(12) Patent Application Publication
(10) Pub. No.: US 2014/0018529 A1
(43) Pub. Date: Jan. 16, 2014

(54) NUCLEIC ACID ISOLATION AND PURIFICATION SYSTEM

(71) Applicant: MICHAEL P. PERRY, Downingtown, PA (US)

(72) Inventor: MICHAEL P. PERRY, Downingtown, PA (US)

(73) Assignee: E I Du Pont De Nemours And Company, Wilmington, DE (US)

(21) Appl. No.: 13/547,078

(22) Filed: Jul. 12, 2012

(51) Int. Cl. B01J 8/06; C07H 1/08

(52) U.S. Cl. 536/25.41; 422/527

(57) ABSTRACT

A device was developed for use in nucleic acid isolation that includes reversibly connected upper and lower chambers, and contains a fibrous nucleic acid binding surface which can expand in the chambers when wetted. The device may be used in a rapid nucleic acid isolation process without clogging while accommodating complex samples.

This application claims the benefit of U.S. Provisional Applications, 61/452,786, 61/452,784, and 61/452,683, all filed on Mar. 15, 2011, all now expired and claim priority to each of U.S. Ser. No. 13/420,150, U.S. Ser. No. 13/420,158, and U.S. Ser. No. 13/420,171, each filed Mar. 14, 2012, now pending.

### FIELD OF INVENTION

The field relates to a device for the isolation of nucleic acids from samples that reduces clogging and may be used in a simple isolation process.

### BACKGROUND OF INVENTION

Isolation and purification of nucleic acids is important for clinical diagnostics and basic molecular and microbiology research such as public health genetic testing, forensics, phylogenetics, transgenics, gene sequencing, and metabolic pathway analysis.

Nucleic acid purification is typically performed via anion exchange on positively charged substrates, or chaotropic capture on silica substrates. A commonly used nucleic acid binding material is a glass surface due to its ability to reversibly bind nucleic acids in the presence of chaotropic reagents and/or alcoholic additives (Boom et al. (1990) J. Clin. Microbiol. 28:495-503). In this type of binding and elution, DNA is “bound” to the negatively charged substrate from very high ionic strength solution (e.g. guanidine thiocyanate), and subsequently eluted in a significantly lower ionic strength solution. These substrates are typically silica fiber filter discs or ion exchange resins that are contained in column type devices that use gravity flow, centrifugation, suction, or pressure from a plunger for nucleic acid isolation. In these types of devices, particulates and/or viscous material (such as extracellular polysaccharides) in a complex sample tend to clog the filter or resin.

WO2011017251 discloses a process for collection of nucleic acids of microorganisms that involves a fibrous nucleic acid binding surface in a chamber where expansion may occur, and compression of the fibrous nucleic acid binding surface to expel fluids.

Many nucleic acid isolation devices and kits that are available require lengthy procedures that include multiple steps and reagents, long incubation times, and complex devices. For example, WO2010075116 discloses a clarification/binding device comprising a clarification column and a binding column, and a vacuum manifold.

There is thus a need for a simple nucleic acid isolation device that can handle relatively large volumes, takes a minimal amount of time, does not clog with particulate or viscous samples, and can be used in a rapid nucleic acid isolation process.

### SUMMARY OF INVENTION

The invention relates to a device can be used for rapid nucleic acid isolation from relatively large volume samples with minimal clogging.

Accordingly, the invention provides a multi-component device for isolating nucleic acids from a sample comprising:
a) a cylindrical upper chamber and a cylindrical lower chamber, said upper and lower chambers detachably connectable at first ends, with said connection affording in its interior a smooth, uniform transition between said upper and lower chambers and at its exterior on the lower chamber a flange, and said lower chamber comprising a screen at its second end;
b) a fibrous nucleic acid binding surface partially filling the inside of the upper and lower chambers and maintained in the chambers by the screen;
c) a removable cap at the second end of the upper chamber and a removable cap at the second end of the lower chamber;
d) a first collection tube into which the lower chamber slides up to the flange wherein an air-tight seal is produced; and
e) an optional second collection tube into which the lower chamber partially slides.

In another embodiment the invention provides a nucleic acid purification kit comprising a device comprising:
a) a cylindrical upper chamber and a cylindrical lower chamber, said upper and lower chambers detachably connectable at first ends, with said connection affording in its interior a smooth, uniform transition between said upper and lower chambers and at its exterior on the lower chamber a flange, and said lower chamber comprising a screen at its second end;
b) a fibrous nucleic acid binding surface partially filling the inside of the upper and lower chambers and maintained in the chamber by the screen;
c) a removable cap at the second end of the upper chamber and a removable cap at the second end of the lower chamber;
d) a first collection tube into which the lower chamber slides up to the flange wherein an airtight seal is produced; and
e) optionally a second collection tube into which the lower chamber partially slides.

In yet another embodiment the invention provides a method of isolating nucleic acids comprising:
a) forming a binding mixture comprising:
   i) a sample containing nucleic acids; and
   ii) a nucleic acid binding solution;
   wherein the binding mixture is either formed within or is added to a double-chamber device comprising:
   iii) a cylindrical upper chamber and a cylindrical lower chamber, said upper and lower chambers detachably connectable at first ends, with said connection affording in its interior a smooth, uniform transition between said upper and lower chambers and at its exterior on the lower chamber a flange, and said lower chamber comprising a screen at its second end;
   iv) a fibrous nucleic acid binding surface partially filling the inside of the upper and lower chambers and maintained in the chambers by the screen; and
   v) a removable cap on the second end of the lower chamber;
b) incubating the binding mixture in the double-chamber device to allow nucleic acid binding;
c) removing the cap and draining the unbound sample by gravity flow;
d) placing the double-chamber device in a first collection tube wherein an air-tight seal is produced;
e) applying a wash solution to the double-chamber device;
f) releasing the seal to drain the wash solution;
g) separating the chambers of the double-chamber device;
h) placing the lower chamber in a centrifuge tube;
i) adding elution buffer to release bound nucleic acid; and
j) centrifuging to collect the elution buffer containing nucleic acid from the sample;
wherein no filtering, plunging, or vacuum step is used.

### BRIEF DESCRIPTION OF THE FIGURES

FIG. 1 shows diagrams of a two-chamber fiber-containing device showing separate components in (A) and a set of assembled components in (B).

FIG. 2 shows diagrams of types of connections to join upper and lower chambers of the two-chamber fiber-containing device: with threads on the upper and lower chambers that screw into a collar as separate components (A) and assembled (B); or with threads on the upper chamber that screw into the lower chamber as separate components (C) and assembled (D).

FIG. 3 shows diagrams of a prototype two-chamber fiber-containing device that uses a modified 3-mL syringe and a modified spin column with original syringe and spin column in (A), modified syringe and spin column in (B), and a set of assembled components, with additional collection microcentrifuge tube in (C).

### DETAILED DESCRIPTION

The invention relates to a device for use in nucleic acid isolation that has reversibly connected upper and lower chambers, and a fibrous nucleic acid binding surface partially filling the upper and lower chambers that can expand in both chambers. The upper chamber allows for a larger volume into which the nucleic acid binding surface can expand than could be accommodated by the lower chamber alone, while after nucleic acid binding, the detached lower chamber can readily be processed in a microcentrifuge. This device can be used in a rapid nucleic acid isolation process without clogging while accommodating complex samples. The resulting isolated nucleic acids are important for clinical diagnostics and basic molecular and microbiology analyses such as public health genetic testing, forensics, phylogenetics, transgenics, gene sequencing, and metabolic pathway analysis.

The following definitions and abbreviations are to be used for the interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 20% of the reported numerical value, preferably within 10% of the reported numerical value, and more preferably within 5% of the reported numerical value.

A “chaotrope” is any chemical substance which disturbs the ordered structure of liquid water. Some examples of the processes that chaotropes facilitate include unfolding, extension, and dissociation of proteins, and hydrogen bonding of nucleic acids. Exemplary chaotropic salts include sodium iodide, sodium perchlorate, guanidinium thiocyanate, guanidinium isothiocyanate, and guanidinium hydrochloride.

The term “isolated” refers to materials, such as nucleic acid molecules and/or proteins, which are substantially removed from components that normally accompany or interact with the materials in a naturally occurring environment. Isolated nucleic acids may be purified from a host cell in which they naturally occur.

The terms “polynucleotide”, “polynucleotide sequence”, “nucleic acid”, “nucleic acid sequence”, and “nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more strands of cDNA, genomic DNA, synthetic DNA, or mixtures thereof. The term also embraces recombinant polynucleotides and chemically synthesized polynucleotides.

The term “silica” as used herein denotes materials which are mainly built up of silicon and oxygen. These materials comprise, for example, silica, silicon dioxide, silica gel, fumed silica gel, diatomaceous earth, celite, talc, quartz, crystalline quartz, amorphous quartz, glass, glass particles including all different shapes of these materials. Glass particles, for example, may comprise particles of crystalline silica, soda-lime glasses, borosilicate glasses, and fibrous, non-woven glass.

The term “expand” as used herein means to spread out to occupy a larger volume. A clump or mass of fibrous material expands, or spreads out, when wetted, such that a larger volume of space is occupied by the fibrous material. The spreading out of fibrous material allows for greater access to surfaces of the fibers. For example, a 10 mg clump of dry fibrous material expands when wetted to into a ˜2-3 ml volume of space.

The term “cylindrical” as used herein includes (1) shapes with sides parallel to each other that are at a fixed distance from a central line from one end to the other of the shape, as well as (2) shapes where the sides form lines that increase in distance from a central line from one end to the other of the shape.

The present device for nucleic acid isolation includes nucleic acid binding material that is fibrous and expands when wetted. Any expandable fibrous material comprising nucleic acid binding surfaces may be used, but preferably, the fibrous nucleic acid binding surface is non-magnetic. In some embodiments this fibrous nucleic acid binding surface is a clean silica surface, with some embodiments utilizing a clean, activated silica surface. Cleaning and activation of the silica is effectuated, e.g., by washing with hydrochloric acid, although a separate cleaning step may not be required for all silica types. In one embodiment the fibrous nucleic acid binding surface is quartz wool, such as Quartzel® wool (4 μm fiber size; Saint-Gobain Quartz, Northboro, Mass.).

Alternatively, the nucleic acid binding surface can be, e.g., NOMEX® fibers (meta-aramid; E.I. du Pont de Nemours & Co., Wilmington, Del.), KEVLAR® (para-aramid; E.I. du Pont de Nemours & Co., Wilmington, Del.), a polyamide, e.g., nylon (e.g., nylon 6,6, nylon 6, nylon 11, nylon 12, nylon 612).

The present device is configured to include components that provide interior space for expansion of the nucleic acid binding fibrous surface that is contained within the device while sample is being applied and washes are performed. The fibrous material is not rigid or packed in the device. Expansion of the nucleic acid binding fibrous material provides greater surface area for nucleic acid binding (as compared to surface available without expansion). In addition, unconfined expansion of the binding material allows suspensions, solutions, and slurries to flow through the material thus avoiding clogging by particulates and/or viscous components of a sample.

The present device includes two chambers, an upper and a lower chamber, that are detachably connected at first ends and that contain the fibrous surface. The upper second end of the upper chamber and the lower second end of the lower chamber have removable caps. Each cap may be held in place in any typical manner such as screwing, snapping, or sliding into or onto the end of the chamber.

Typically the connected chambers form a cylindrically shaped tube, although the diameter of the tube, particularly the inside diameter, may decrease from the upper end of the upper chamber to the lower end of the lower chamber. The lower chamber may also have a narrowed second end. This lower chamber is of size that is readily partially inserted at its second end into a 1.5 to 2.0 milliliter second collection tube. Preferably the second collection tube is a centrifuge tube, and the centrifuge tube with the lower chamber partially inserted is amenable to centrifugation. Preferably the second collection tube fits into a standard microcentrifuge. The second collection tube is optionally part of the device.

A screen at the lower end of the lower chamber retains the nucleic acid binding fibrous surface inside the chamber at this end during sample and wash applications, and centrifugation. The screen may be any type of screen that can retain the nucleic acid binding fibrous surface, for example, a screen that is woven, extruded, wire, or polypropylene, which may be a separate component or may be molded as part of the lower chamber itself. In one embodiment the screen is formed by molded perforations in the lower end of the chamber. The screen allows material in a sample and washes to pass through while retaining the nucleic acid binding fibrous surface. The screen remains relatively free of clogging from particulates and viscous material in a sample, as compared to a silica fiber disc, filter, or ion exchange resin such as those typically used in nucleic acid isolation.

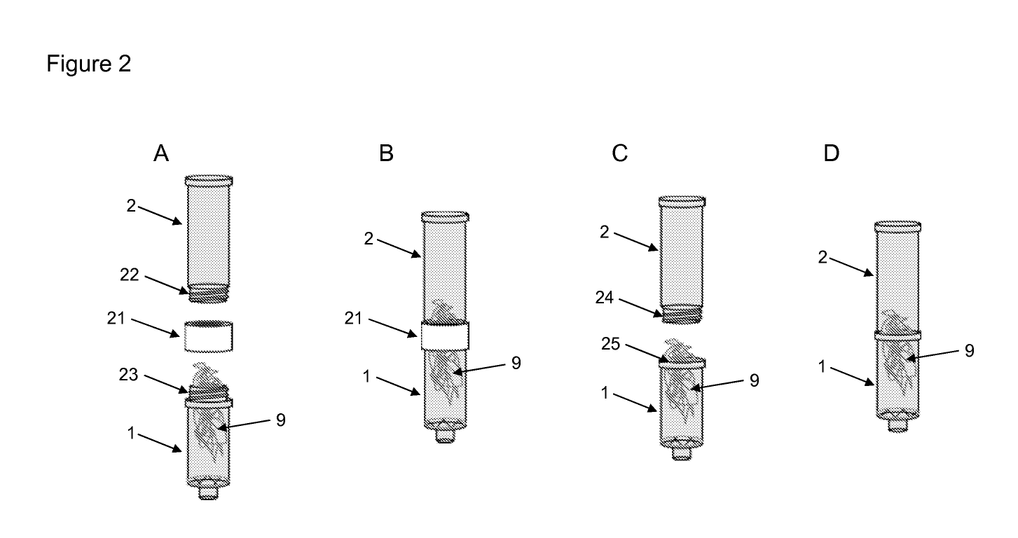

The upper chamber detachably connects to the lower chamber, at first ends of each chamber, with a smooth uniform transition of the interiors of the two chambers. The uniform transition allows the nucleic acid binding fibrous surface to expand unhindered into both chambers when it is wetted. Any type of connection that allows attachment and detachment of the two chambers, and that forms an internal uniform transition, may be used, including for example screw and snap connections. Examples of different types of detachable connections that may be used are described below with reference to FIG. 2.

The lower chamber also inserts at its second end into a first collection tube for collecting the sample and washes that pass through the screen. A flange on the exterior at the upper end of the lower chamber rests on the top open end of the collection tube. The flange can be separate from, or part of, a mechanism that connects the lower chamber with the upper chamber. Preferably, the lower chamber fits tightly in the first collection tube forming an air-tight seal. The tight fit may be accomplished by any effective structure such as from a flange at the top of the lower chamber that fits tightly into the collection tube, or the outside diameter at the top of the lower chamber and inside diameter of the collection tube being such that a tight fit is achieved. With this seal, sample and washes that are loaded into the upper and lower connected chambers remain in these chambers until the seal is broken. The first collection tube typically has a volume capacity that is at least as large as the capacity of the combined upper and lower chambers, so that for example the complete volume of a wash loaded into the connected chambers may be collected in the tube.

The present device does not contain any filters and there is no filtering of an applied sample. The present device does not make use of a plunger, such as one typically used in a syringe or other forced air mechanism. The present device does not attach to a vacuum-creating device and there is no vacuum applied to move a sample through the device.

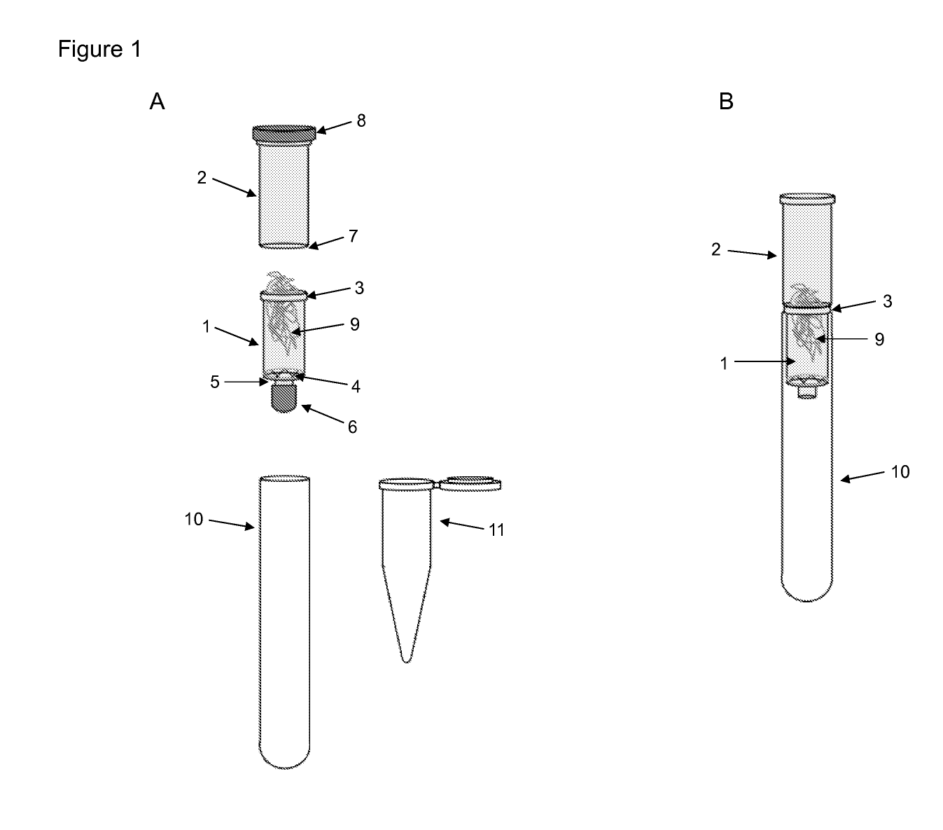

An embodiment of the present device is diagrammed in FIG. 1. In FIG. 1 (A) a lower chamber (1) and an upper chamber (2) of the device are shown detached from each other. The two attachable chambers are cylindrical and of the same internal diameter. The lower chamber has an external flange (3) at the first end, which is the attaching end, and a screen (4) at the second end. In addition the second end of the lower chamber is narrowed (5) and has a removable cap (6). The upper chamber has an attaching first end (7) and a second end with a removable cap (8). A fibrous nucleic acid binding surface (9) partially fills the inside of the lower chamber and is maintained in the chamber lower end by the screen (4). A first collection tube (10) is slightly larger in diameter than the lower chamber. The first collection tube has a capacity of about 5 ml. A second collection tube (11) is slightly larger in diameter than the lower chamber in the top section and is in the form of a microfuge tube. In FIG. 1(B) an assembled device with the first collection tube is shown. The upper chamber (2) and the lower chamber (1) of FIG. 1(A) are connected, and the fibrous nucleic acid binding surface (9) partially fills the lower and upper chambers. The lower chamber is inserted into the first collection tube (10) with the flange (3) at the first end of the lower chamber resting on the top open end of the first collection tube. The lower chamber is designed to fit snuggly into the first collection tube so as to create an airtight seal to prevent fluid flow from the connected upper and lower chambers into the collection tube when assembled as in FIG. 1(B).

Two embodiments of detachable connections of the lower (1) and upper (2) chambers containing a fibrous nucleic acid binding surface (9) are diagrammed in FIG. 2. The connection in FIG. 2(A) consists of an internally threaded collar section (21) into which external threads on the first end of the upper chamber (22) and external threads on the first end of the lower chamber (23) screw. The device with assembled upper and lower chambers with the collar connection is shown in FIG. 2(B). The connection in FIG. 2(C) is a simple screw connection consisting of external threads on the first end of the upper chamber (24) that screw into internal threads in the first end of the lower chamber (25). The device with assembled upper and lower chambers with the simple screw connection is shown in FIG. 2(D).

### Nucleic Acid Extraction

The present device is used to isolate nucleic acids from a sample. Any sample may be used that contains nucleic acids, such as DNA or RNA, in a suspension, solution, or slurry. Examples of samples that may be used include environmental samples, culture samples, tissue samples, and serum samples, from which nucleic acids may be prepared for analysis such as for clinical, environmental, or research analysis. Nucleic acids may be naturally accessible in the sample, or treatments may be used to gain accessibility to the nucleic acids prior to loading a sample. Treatments may include any that disrupt cells such as sonication, heat, chemical and/or enzymatic, that result in cell lysis.

Typically, the volume of sample may range from 500 μL to 5 mL or higher depending on sample type and volume of the upper chamber. When DNA is the targeted nucleic acid for extraction, RNase may be added to digest RNA in the sample. An equal volume of nucleic acid binding solution is typically added to the sample prior to transferring, or loading, into the upper chamber. The binding solution contains components such as detergent, ethylenediaminetetraacetic acid (EDTA), guanidinium thiocyanate and/or other components that facilitate binding. The binding solution may also contain other components that facilitate cell lysis. The nucleic acid binding solution is typically a chaotropic salt but alternatively can be a blend of salts such as 6 M NaClO₄, Tris, and trans-1,2-cyclohexanediaminetetraacetic acid (CDTA); or 8 M NaClO₄ and Tris at pH 7.5; or NaI.

A typical sample processing method is as follows. The binding solution and sample are mixed and the resulting binding mixture is transferred into the double chamber fiber-containing column of the device, with a cap in place on the lower end. Upon wetting, the fibrous nucleic acid-binding surface in the column disperses and expands in the chambers to expose the bulk of its surface area. The device is then incubated during which time cell lysis (if cells are present) and nucleic acid binding occur. Then the cap is removed and the binding solution and unbound sample drain from the column by gravity flow. The fibrous nucleic acid binding surface remains in the column.

With the column tightly seated in a 5 mL collection tube, a wash solution is transferred to the column and allowed to sit for up to a few minutes. The wash solution does not appreciably release nucleic acids bound to the fiber surface, while removing undesired substance. Typical wash solutions are buffers with low or moderate ionic strength such as, for example, 10 mM Tris-HCl at a pH of 8, 0-10 mM NaCl, or acetone or alcohol containing media such as, for example, solutions of lower alkanols with one to five carbon atoms, typically ethanol in water such as aqueous 70% ethanol, or isopropanol. The seating is released and the wash solution drains into the collection tube. The fibrous nucleic acid binding surface remains in the column. Washing may be repeated.

The upper and lower chambers of the column are then separated. The lower chamber containing the fibrous nucleic acid binding surface is placed in a 1.5 to 2-mL microcentrifuge tube and centrifuged briefly to remove liquid from the fiber surface. The lower chamber is moved to a clean 1.5-mL microcentrifuge tube and an elution buffer, typically about 50-500 μl, is transferred onto the fiber in the chamber. Typical elution buffers are water or a dilute Tris-HCl solution, pH 8.5.

After exposure of the fibrous material to the elution buffer for a few minutes, the elution buffer containing nucleic acids that previously were bound to the nucleic acid-binding surface is collected by centrifugation. As necessary, additional elution buffer can be added into the device, mixed with the nucleic acid-binding surface, and collected to maximize recovery of bound nucleic acids.

Also, in the elution step, a stabilizing agent for preventing degradation of nucleic acid recovered in the elution solution of nucleic acid may be included. Examples of stabilizing agents include an antibacterial agent, a fungicide, and a nucleic acid degradation inhibitor such as EDTA.

### EXAMPLES

The present invention is further defined in the following Examples. It should be understood that these Examples, while indicating preferred embodiments of the invention, are given by way of illustration only.

The meaning of abbreviations is as follows: “h” means hour(s), “min” means minute(s), “sec” means second(s), “d” means day(s), “mL” means milliliter(s), “μL” means microliter(s), “um” means micron(s), “mm” means millimeter, “mM” is millimolar, “M” means molar, “CFU” means colony forming unit, “mg” means milligrams, “EDTA” means ethylenediaminetetraacetic acid.

### General Methods

### Prototype Double Chamber with Fiber Device

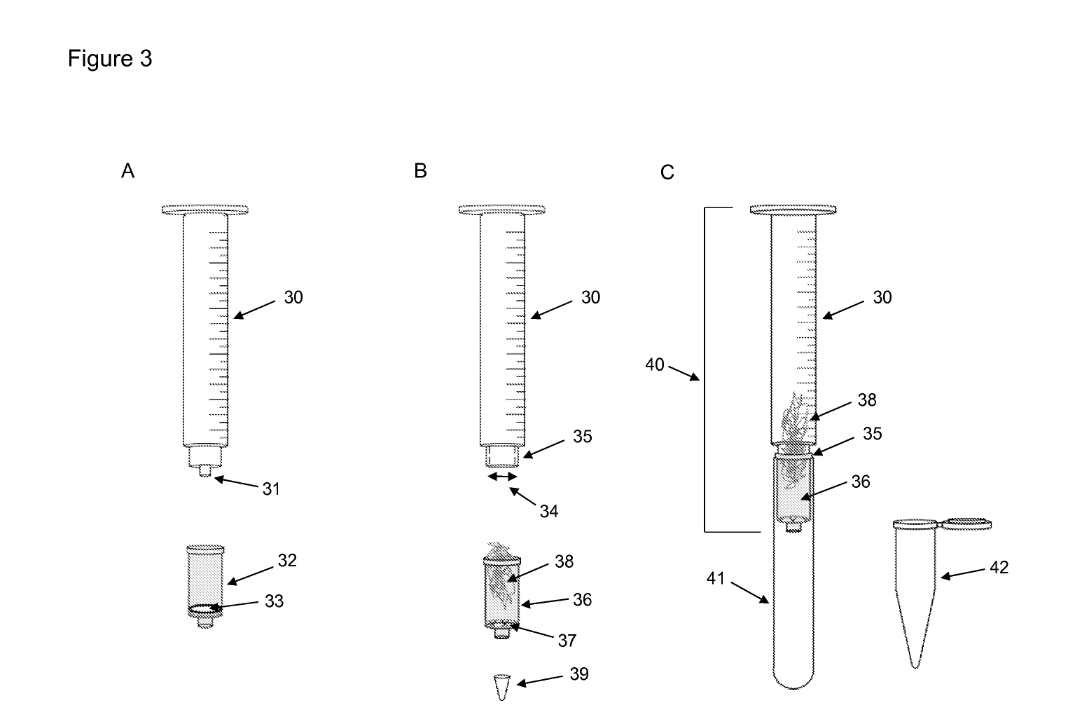

A prototype device having a double chamber column with fibrous nucleic acid binding surface was assembled as shown in FIGS. 3 (A, B, C) using a modified 3 ml syringe barrel (30) for the upper chamber. The tip of the syringe (FIG. 3(A): 31) was drilled out to 5/16 inch (7.9 mm; FIG. 3(B): 34) at the narrow end of the syringe barrel (FIG. 3(B): 35). A QIAGEN QIAprep spin column (#1018398, QIAGEN, Inc., Valencia, Calif.) (FIG. 3(A): 32), with the silica membrane (33) at the bottom end removed, was used for the lower chamber (FIG. 3(B): 36). The silica membrane was replaced with a polypropylene mesh screen (37) and 10 mg of Quartzel® wool of 4 μm fiber size (FIG. 3(B): 38) (Saint-Gobain Quartz, Northboro, Mass.) was placed into the device. The plunger for the 3 ml syringe was used to cap the upper end. The bottom tip of a thin walled 0.65 mL PCR tube was cut off and used as the lower cap (FIG. 3(B): 39). The narrow end of the syringe (FIG. 3(B) 35) fit tightly into the top open end of the modified QIAprep spin column (FIG. 3(C): 36) forming a double chamber column (FIG. 3(C): 40) with the Quartzel® wool fiber partially filling both chambers of the column (FIG. 3(C): 38). A 5 mL round bottom tube (Falcon #352063, Becton Dickinson, Franklin Lakes, N.J.) (FIG. 3(C): 41) was used as a processed sample and wash collection tube. The modified QIAprep spin column fit tightly into the open end of this test tube. A 1.5 mL microfuge tube (#022363204, Eppendorf North America, Hauppauge, N.Y.) (FIG. 3(C): 42) was used as a nucleic acid collection tube. The modified QIAprep spin column also fits into the open end of this microfuge tube.

### Example 1

### DNA Isolation Using a Prototype Device

The double chamber fiber-containing device described in General Methods was used to isolate DNA as follows. Shewanella putrefaciens, strain LH4:18 (ATCC No. PTA-8822) was grown overnight in simulated injection brine (SIB: 198 mM NaCl, 1 mM MgCl₂, 1.8 mM CaCl₂, 1.2 mM KCl, 16 mM NaHCO₃, 0.05 mM SrCl₂, 0.13 mM BaCl₂, 0.14 mM LiCl) supplemented with 1% peptone. Cells were diluted to 1×10⁹ CFU/mL in LB. One milliliter of the diluted cell suspension was treated with 10 μL of 10 mg/mL RNase A for 5 min at room temperature. Another 1 mL of the diluted culture received no RNase treatment. To each of the dilution samples, 1 mL of L6 Lysis/Binding Buffer (5 M Guanidinium thiocyanate, 50 mM Tris-Cl pH 6.4, 22 mM EDTA, 1.3% Triton X-100) was added. The 2-mL samples were vortexed and pipetted into the double chamber fiber-containing column, capped at the lower end, onto the Quartzel® wool. The column was then capped at the upper end with the syringe plunger and at the lower end with the lower cap. The column was briefly shaken to disperse the fiber and was incubated at room temperature for 5 min to allow DNA in the samples to bind to the quartz fiber. During this time the wool fiber binding surface expanded in the double chamber column. The caps were removed, the lower chamber of the column set into the 5 ml test tube, and the liquid was allowed to passively flow through the double chamber column and into the collection tube. The flow through was discarded and the column was reseated snuggly into the collection tube. Two and a half milliliters of isopropanol (IPA) were pipetted into the device and allowed to sit for 1 min. The column was unseated from the collection tube and the liquid was allowed to passively flow through the device and into the collection tube. The flow through was discarded and the IPA wash was repeated. The syringe barrel was then removed and discarded. The lower spin column containing the fiber was placed into a 2 mL centrifugation tube and the fiber was dried by centrifuging for 1 min at 16000×g. The spin column was then placed into a clean 1.5 mL tube and 50 μL of elution buffer (10 mM Tris, pH 8.5) was added to the fiber and incubated for 5 min to release fiber-bound DNA. The DNA was recovered by centrifugation for 30 sec at 10000×g. The elution step was repeated.

Five microliters of the recovered nucleic acids were analyzed by electrophoresis on a 1% agarose gel stained with ethidium bromide, with an E. coli DNA sample used as a control. The preparation yielded a high molecular weight ethidium bromide-stained band on the gel, representing DNA with molecular weight greater than that of the E. coli DNA. There was more recovered DNA in the RNase treated sample than in the untreated sample.

### Example 2

### Comparison of Double-Chamber Fiber-Containing Device and Qiagen DNeasy Blood and Tissue Kit

Pseudomonas stutzeri, strain BR5311 (ATCC No. PTA 11283) was grown overnight in LB. Cells were serially diluted by factors of ten from 1×10⁸ CFU/mL down to 1×10⁵ CFU/mL. One milliliter aliquots of the dilutions were run in QIAGEN's DNeasy Blood and Tisssue Kit (QIAGEN, Inc., Valencia, Calif.), following the supplier's protocol. Samples were processed using the double chamber fiber-containing device described in General Methods as follows. One milliliter of each dilution was treated with 10 μL of 10 mg/mL RNase A for 5 min at room temperature. One milliliter of L6 Lysis/Binding Buffer was added. The 2-mL samples were vortexed and pipetted into the double chamber fiber-containing column and processed as described in Example 1. Twenty microliters of the recovered samples were run on a 1% agarose gel stained with ethidium bromide. DNA recovered from isolation using the double chamber fiber-containing column was similar to that recovered from the QIAGEN DNeasy Blood and Tisssue Kit; both processes detected DNA from samples having 1×10⁷ CFU/mL, as the lower limit of detection. It should also be noted that the process using the double chamber fiber-containing device was completed in its entirety during the one hour incubation step required for the QIAGEN procedure, which then requires an additional 40-45 minutes to complete.

### Example 3

### Comparison of Double-Chamber Fiber-Containing Device and Qiagen DNeasy Blood and Tissue Kit for DNA Isolation from Shewanella

When lysed, Shewanella bacteria release large quantities of nucleic acids, extracellular polymeric substances, and other cellular debris, more so than typical Gram negative bacteria. Solutions of lysed Shewanella cells, therefore, become quite viscous and are difficult to work with. Using both kit (QIAGEN Genomic-tip 100/G) and phenol extraction based DNA isolation procedures, we had been attempting to isolate genomic DNA from these strains of bacteria. But, due to the general nature and increased viscosity of the samples, these attempts proved unsuccessful. To test the efficacy of the double chamber fiber-containing prototype device on a viscous, troublesome sample, we compared isolation using the prototype device with QIAGEN's DNeasy Blood and Tisssue Kit procedure, as in Example 2 above.

Shewanella, strain MPHPW-1 (ATCC No. PTA-11920) was grown overnight in LB. Cells were diluted to 2×10⁹ CFU/mL. One milliliter aliquots of the dilutions were run in QIAGEN's DNeasy Blood and Tisssue Kit, following the supplier's protocol. Samples were processed using the double chamber fiber-containing device described in General Methods as follows. One milliliter aliquots of each dilution were treated with 10 μL of 10 mg/mL RNase A for 5 min at room temperature. One milliliter of L6 Lysis/Binding Buffer was added to each. The 2-mL samples were vortexed and pipetted into the double chamber fiber-containing column device and processed as described in Example 1. Twenty microliters of the samples were run on a 1% agarose gel stained with ethidium bromide. Based on the intensity of band staining on the gels, more DNA was isolated using the double chamber fiber-containing device than from the QIAGEN DNeasy Blood and Tisssue Kit. It should also be noted that the entire procedure using the double chamber fiber-containing device was completed in less than half the time as used for the QIAGEN procedure.

### Example 4

### Test of Reproducibility of the Prototype Device

To test reproducibility of DNA isolation using the double chamber fiber-containing device, we lysed an aliquot of a culture of Shewanella cells and split it across four of the double chamber fiber-containing columns. Forty microliters of 10 mg/mL RNase A was added to 4 mL of Shewanella strain MPHPW-1 cells (approximately 6.7×10⁸ CFU/mL in LB) and the sample was incubated for 5 min at room temperature. An equal volume of L6 Lysis/Binding Buffer was added, the sample was vortexed, and 2 mL samples were loaded onto 4 double chamber fiber-containing columns. The isolation procedure was followed as described in Example 1.

Twenty microliters of the samples were run on a 1% agarose gel stained with ethidium bromide. Stained bands of similar intensities were observed on the gel. Optical densities of the recovered DNA samples were also measured and used to determine amounts of DNA recovered, which are given in Table 1. Similar amounts of DNA were recovered from the 4 samples.

TABLE 1
OD₂₆₀ readings and calculated DNA recoveries

| Sample # | OD₂₆₀ | DNA recovered (μg) |
|---|---|---|
| 1 | 2.64 | 26.4 |
| 2 | 2.94 | 29.4 |
| 3 | 3.25 | 32.5 |
| 4 | 3.01 | 30.1 |

### Example 5

### Isolation of DNA from Gram Positive Bacteria

To isolate genomic DNA from the Gram positive bacteria Bacillus subtilis (ATCC #82) and Marinococcus albus (ATCC #49811) a lysozyme and proteinase K digestion step was added to the process used in Example 1 to ensure complete cell lysis. B. subtilis was grown overnight in Miller's LB Medium (Mediatech, Inc., Manassas, Va.) and M. albus was grown in Marine Broth 2216 (Becton Dickinson Co., Franklin Lakes, N.J.). One milliliter aliquots of the overnight cultures were centrifuged in a microcentrifuge at maximum speed for 3 min. The cell pellets were resuspended in 1 mL of Lysis Buffer (100 mM Tris, 50 mM NaCl, 50 mM EDTA, pH 8.0). To each sample, 100 μL of 20 mg/mL lysozyme, 100 μL of 10% SDS, 100 μL of 10% sarkosyl, 10 μL of 10 mg/mL RNase, and 10 μL of 10 mg/mL proteinase K were added. Samples were vortexed and incubated at 37° C. for 45 min. One milliliter L6 Buffer was added and the samples were pipetted into the double chamber fiber-containing column and processed as described in Example 1. Twenty microliters of the recovered DNA samples were run on a 1% agarose gel stained with ethidium bromide. Stained bands of molecular weight representing bacterial DNA were observed which showed that with the lysozyme and proteinase K additions, DNA isolation from Gram positive bacteria using the double chamber fiber-containing prototype device was effective.

### Example 6

### Isolation of DNA from Rat Tissues

The effectiveness of the double-chamber fiber-containing device for the isolation of genomic DNA from tissue samples was tested. In order to ensure complete digestion of the samples, the procedure used included a lysozyme and proteinase K digestion step. One milliliter of Lysis Buffer (100 mM Tris, 50 mM NaCl, 50 mM EDTA, pH 8.0) was added to 50 mg each of rat brain, lung, tail, and heart tissue samples. To each sample, 100 μL of 20 mg/mL lysozyme, 100 μL of 10% SDS, 100 μL of 10% sarkosyl, 10 μL of 10 mg/mL RNase, and 10 μL of 10 mg/mL proteinase K were added. The samples were vortexed and incubated at 37° C. for 30 min. One milliliter L6 Buffer was then added and the samples were vortexed and incubated again for 30 min at 37° C. The samples were then pipetted into the double chamber fiber-containing column and processed as described in Example 1. Twenty microliters of the samples were run on a 1% agarose gel stained with ethidium bromide. Stained bands of molecular weight representing rat genomic DNA were observed which showed that with the lysozyme and proteinase K digestion step, the double chamber fiber-containing prototype device was effective for isolation of DNA from mammalian tissues of various types.

What is claimed is:

1. A multi-component device for isolating nucleic acids from a sample comprising:
a) a cylindrical upper chamber and a cylindrical lower chamber, said upper and lower chambers detachably connectable at first ends, with said connection affording in its interior a smooth, uniform transition between said upper and lower chambers and at its exterior on the lower chamber a flange, and said lower chamber comprising a screen at its second end;
b) a fibrous nucleic acid binding surface partially filling the inside of the upper and lower chambers and maintained in the chambers by the screen;
c) a removable cap at the second end of the upper chamber and a removable cap at the second end of the lower chamber;
d) a first collection tube into which the lower chamber slides up to the flange wherein an air-tight seal is produced; and
e) an optional second collection tube into which the lower chamber partially slides.

2. The device of claim 1 wherein the nucleic acid-binding surface is a silica surface.

3. The device of claim 2 wherein the silica surface comprises quartz wool.

4. The device of claim 1 further comprising a narrowed second end of the lower chamber.

5. The device of claim 1 wherein the upper and lower chambers are detachably connected with a collar.

6. The device of claim 1 wherein the upper and lower chambers are detachably connected by screwing or snapping the upper chamber to the lower chamber.

7. The device of claim 1 wherein the fibrous nucleic acid binding surface expands in both upper and lower chambers when wetted.

8. The device of claim 1 wherein the first collection tube has a volume capacity that is at least as large as the capacity of the combined upper and lower chambers.

9. The device of claim 1 wherein the second collection tube containing the detached lower chamber is amenable to centrifugation.

10. A nucleic acid purification kit comprising the device of claim 1.

11. A method of isolating nucleic acids comprising:
a) forming a binding mixture comprising:
i) a sample containing nucleic acids; and
ii) a nucleic acid binding solution;
wherein the binding mixture is either formed within or is added to a double-chamber device comprising:
iii) a cylindrical upper chamber and a cylindrical lower chamber, said upper and lower chambers detachably connectable at first ends, with said connection affording in its interior a smooth, uniform transition between said upper and lower chambers and at its exterior on the lower chamber a flange, and said lower chamber comprising a screen at its second end;
iv) a fibrous nucleic acid binding surface partially filling the inside of the upper and lower chambers and maintained in the chambers by the screen; and
v) a removable cap on the second end of the lower chamber;
b) incubating the binding mixture in the double-chamber device to allow nucleic acid binding;
c) removing the cap and draining the unbound sample by gravity flow;
d) placing the double-chamber device in a first collection tube wherein an air-tight seal is produced;
e) applying a wash solution to the double-chamber device;
f) releasing the seal to drain the wash solution;
g) separating the chambers of the double-chamber device;
h) placing the lower chamber in a centrifuge tube;
i) adding elution buffer to release bound nucleic acid; and
j) centrifuging to collect the elution buffer containing nucleic acid from the sample;
wherein no filtering, plunging, or vacuum step is used.
